(19) United States
(12) Patent Application Publication
(10) Pub. No.: US 2014/0275535 A1
(43) Pub. Date: Sep. 18, 2014

(54) ACID ADDITION SALTS OF BOSENTAN

(71) Applicants: Srinivas Pullela Venkata, Andhra Pradesh (IN); Kiran Kumar Kothakonda, Karnataka (IN); Shanmughasamy Rajmahendra, Chennai (IN); Indrajit Chandrasekaran, Trichy (IN); Mariappan Kaliappan, Madurai (IN); Rekha Shivappa Mailar, Karnataka (IN)

(72) Inventors: Srinivas Pullela Venkata, Andhra Pradesh (IN); Kiran Kumar Kothakonda, Karnataka (IN); Shanmughasamy Rajmahendra, Chennai (IN); Indrajit Chandrasekaran, Trichy (IN); Mariappan Kaliappan, Madurai (IN); Rekha Shivappa Mailar, Karnataka (IN)

(73) Assignee: Biocon Limited, Bangalore (IN)

(21) Appl. No.: 14/352,237

(22) PCT Filed: Dec. 1, 2011
(86) PCT No.: PCT/IB2011/055409

§ 371 Date: Apr. 16, 2014

(30) Foreign Application Priority Data
Oct. 18, 2011 (IN) 3581/CHE/2011

(51) Int. Cl. C07D 239/52; C07C 59/265; C07C 59/255

(52) U.S. Cl. CPC C07D 239/52; C07C 59/255; C07C 59/265
USPC 544/296

(57) ABSTRACT

The present invention relates to the stable acid addition salts of Bosentan that are useful for the purification of Bosentan base. In particular, the Bosentan acid addition salt is selected from Bosentan citrate and Bosentan tartrate.

### TECHNICAL FIELD

The present invention describes an efficient process for the preparation of salts of Bosentan and isolation of substantially pure base thereof and also the isolation of substantially pure crystalline intermediates involved in the process.

### BACKGROUND AND PRIOR ART OF THE INVENTION

The present invention relates to acid addition salts of Bosentan, methods of purifying Bosentan base using the salts and amorphous Bosentan. The acid addition salts are useful for the purification of Bosentan base. Further, the acid addition Bosentan salts of the invention can be obtained in a stable solid-state form making them useful for purification and bulk storage.

Bosentan is chemically known as 4-(1,1-Dimethylethyl)-N-[6-(2-hydroxyethoxy)-5-(2- methoxyphenoxy)[2,2′-bipyrimidin]-4-yl]benzenesulfonamide, having the structural formula-

[chemical structure]

Bosentan is a pharmaceutically active compound (an endothelin receptor antagonist) useful for the treatment of pulmonary arterial hypertension and is represented by the formula (I). Bosentan and its analogues as potential endothelin inhibitors have been first disclosed in U.S. Pat. No. 5,292,740. The patent also disclosed the methods for preparing these compounds. One of the methods involves the condensation of diethyl (2-methoxyphenoxy) malonate with pyrimidine-2-carboxyamidine in the presence of sodium methoxide, to provide the dihydroxy derivative, which is converted into dichloro derivative by the treatment with refluxing phosphorus oxychloride. One of the chlorine of the dichloro derivative is replaced by 4-tert- butylbenzenesulfonamide. The remaining chlorine is replaced by ethylene glycol using monosodium ethylene glycolate to provide Bosentan as illustrated in Scheme-1.

[chemical structure]

[chemical structure]

[chemical structure]

### STATEMENT OF THE INVENTION

Accordingly, the present invention relates to acid addition salts of Bosentan; a method of making an acid addition salt of Bosentan, which comprises: a) combining Bosentan base and an acid having a pKa higher than 3 in an organic solvent to form a solution; b) precipitating a Bosentan acid addition salt from said solution and c) isolating the precipitated Bosentan acid addition salt; a method of purifying Bosentan, which comprises: a) preparation of an acid addition salt of Bosentan by following any method discussed in earlier claims, b) isolation of said acid addition salt of Bosentan from organic solvent c) optionally re crystallizing by using an organic solvent d) converting Bosentan acid addition salt into Bosentan base and e) isolation of Bosentan base; a process which comprises dissolving a solid Bosentan acid addition salt as above in an organic solvent, and precipitating said salt to obtain a purified solid Bosentan acid addition salt; Bosentan citrate salt; and Bosentan tartarate salt.

### BRIEF DESCRIPTION OF THE ACCOMPANYING FIGURES

In order that the invention may be readily understood and put into practical effect, reference will now be made to exemplary embodiments as illustrated with reference to the accompanying figures. The figure together with a detailed description below, are incorporated in and form part of the specification, and serve to further illustrate the embodiments and explain various principles and advantages, in accordance with the present disclosure where:

FIG. 1 is an XRPD pattern for the crystalline Bosentan citrate salt.

FIG. 2 is an XRPD pattern for the crystalline Bosentan tartarate salt.

### DETAILED DESCRIPTION OF THE INVENTION

The present invention relates to acid addition salts of Bosentan.

In an embodiment of the present invention the said acid has a pKa higher than 3.

In another embodiment of the present invention the said salts are in solid form.

In yet another embodiment of the present invention the said salts are in crystalline form.

In still another embodiment of the present invention the said acid addition salt is selected from Bosentan citrate and Bosentan tartarate.

In still another embodiment the said salt is selected from crystalline Bosentan citrate and crystalline Bosentan tartarate.

The present invention further relates to a method of making an acid addition salt of Bosentan, which comprises: a) combining Bosentan base and an acid having a pKa higher than 3 in an organic solvent to form a solution; b) precipitating a Bosentan acid addition salt from said solution and c) isolating the precipitated Bosentan acid addition salt.

In an embodiment the said acid is selected from the group of citric acid and tartaric acid.

In another embodiment the said organic solvent is selected from the group consisting of ketones, chlorinated hydrocarbons, hydrocarbons, alcohols, esters; aliphatic nitriles; ethers and mixtures thereof.

In yet another embodiment the said organic solvent is selected from the group consisting of acetone, methyl tert.butyl ketone, dichloromethane, methanol, ethanol, isopropanol, ethyl acetate, butyl acetate, acetonitrile, di-isopropyl ether, methyl tertiary butyl ether tetrahydrofuran, and mixtures thereof.

The present invention further relates to a method of purifying Bosentan, which comprises: a) preparation of an acid addition salt of Bosentan by following any method discussed in earlier claims, b) isolation of said acid addition salt of Bosentan from organic solvent c) optionally re crystallizing by using an organic solvent d) converting Bosentan acid addition salt into Bosentan base and e) isolation of Bosentan base.

In an embodiment the Bosentan base is amorphous in nature

In another embodiment the said organic solvent is selected from the group consisting of ketones, chlorinated hydrocarbons, hydrocarbons, alcohols, esters, aliphatic nitrites, ethers and mixtures thereof.

In yet another embodiment said organic solvent is selected from the group consisting of acetone, methyl tert.butyl ketone, dichloromethane, methanol, ethanol, isopropanol, ethyl acetate, butyl acetate, acetonitrile, di-isopropyl ether, methyl tertiary butyl ether tetrahydrofuran, and mixtures thereof.

In still another embodiment said converting step comprises contacting said Bosentan acid addition salt with an organic or inorganic base in organic solvent.

In still another embodiment the method further comprises recrystallizing said isolated acid addition salt of Bosentan prior to said converting step.

The present invention further relates to a process which comprises dissolving a solid Bosentan acid addition salt as above in an organic solvent, and precipitating said salt to obtain a purified solid Bosentan acid addition salt.

The present invention further relates to Bosentan citrate salt.

The present invention further relates to Bosentan tartarate salt.

The present invention relates to the discovery of stable acid addition salts of Bosentan that are useful for the purification of Bosentan base. Accordingly, a first aspect of the invention relates to an acid addition salt of Bosentan, wherein said salt is in the solid state and wherein said acid has a pKa values higher than 3. The acid can be selected from organic acids preferably tartaric acid and citric acid. The solid form obtained is crystalline in nature. In particular, the Bosentan acid additional salt is selected from Bosentan citrate and Bosentan tartarate, preferably from crystalline Bosentan citrate and crystalline Bosentan tartarate.

Yet another aspect of the present invention relates to a method of making an acid addition salt of Bosentan, which comprises combining Bosentan base and an acid having a pKa higher than 3 in an organic solvent, preferably a polar organic solvent, to form a solution; precipitating a Bosentan acid addition salt from said solution; and optionally purifying the precipitated Bosentan acid addition salt.

A further aspect of the present invention relates to a method of purifying Bosentan, which comprises combining crude Bosentan and an acid having a pKa more than 3 in a first solvent to obtain an acid addition salt of Bosentan; isolating said acid addition salt of Bosentan from said first solvent; optionally purifying the acid addition salt in the second solvent, converting said pure Bosentan acid addition salt into Bosentan base in a third solvent; and isolating said Bosentan base from said third solvent.

Another aspect of the invention relates to a process of purifying Bosentan salt that comprises dissolving a solid Bosentan acid addition salt in an organic solvent and precipitating said salt to obtain a purified solid Bosentan acid addition salt.

Another aspect of the invention is to provide the crystalline form of the Bosentan Intermediates 5-(2-methoxyphenoxy) [2,2]-bipyrimidinyl diol compound of formula-2, 4,6- dichloro 5-(2-methoxyphenoxy) [2,2]-bipyrimidinyl compound of formula-3, 4-tert-Butyl-N- [6-chloro5-(2-methoxyphenoxy) [2,2]-bipyrimidinyl-4-yl]-benzene sulfonamide compound of formula-4, 4-tert-butyl-N-[6-(2-hydroxyethoxy)-5-(2-methoxyphenoxy)-2-(pyrimidin-2- yl)pyrimidin-4-yl]benzene-1-sulfonamide citrate salt compound of formula-5, 4-tert-butyl-N- [6-(2-hydroxyethoxy)-5-(2-methoxyphenoxy)-2-(pyrimidin-2-yl)pyrimidin-4-yl]bhenzene-1-sulfonamide citrate salt compound of formula-6 in the solid state with good yield, high purity and ease of operations. The intermediate is further characterized by well established techniques like XRD, DSC and FTIR.

[chemical structure]

[chemical structure]

In another embodiment, the present invention provides a crystalline form of Formula-2 having the X-ray diffraction pattern with peaks at 9.815, 11.685, 11.980, 12.587, 14.664, 15.016, 16.261, 19.591, 20.435, 21.387, 22.371, 23.412, 24.184, 24.904, 25.312, 25.944, 26.591, 27327, 28.047, 28.708, 29.139, 29.605, 30.384, 30.896, 31.220, 32.339, 32.717, 33.381, 34.113, 34.511, 34.958, 35.399, 35.92, 37.188, 37.768, 38.340, 39.323, 39.838, 40.197, 42.013, 43.466, 44.384, 45.412, 46.138, 47.837, 48.344, 49.542, 50.932, 51.866, 52.967, 54.077, 54.830, 58.510, 59.565, ±0.2 degrees two theta values.

In another embodiment the present invention provides FTIR spectra of crystalline form of Formula-2 with peaks at 3254, 3151, 2842, 1796, 1663, 1619, 1603, 1578,1565, 1516, 1501, 1472, 1448, 1434, 1402, 1357, 1322, 1298, 1253, 1232, 1213, 1180, 1166, 1156, 1148, 1114, 1088, 1058, 1052, 1022, 946, 835, 817, 807, 763, 723, 713, 687, 677, 644, 635, 598, 555, 506, 458 cm-1.

In another embodiment the present invention provides DSC thermogram of crystalline form of Formula-2 with endothermic peak at 170.34° C.

In another embodiment, the present invention provides a crystalline form of Formula-3 having the X-ray diffraction pattern with peaks at 6.394, 8.610, 9.877, 11.068, 11.969, 13.907, 14.752, 15.547, 17.255, 18.485, 19.824, 20.473, 22.213, 22.718, 23.577, 24.106, 24.823, 25.893, 26.307, 26.578, 27.295, 27.532, 27.790, 28.211, 29.315, 30.064, 30.792, 31.490, 32.028, 32.415, 33.594, 34.273, 34.851, 35.208, 35.681, 36.444, 37.417, 38.545, 39.016, 40.266, 40.638, 41.551, 41.918, 43.093, 43.458, 43.811, 44.393, 45.265, 47.350, 45.995, 49.012, 49.426, ±0.2 degrees two theta values.

In another embodiment the present invention provides FTIR spectra of crystalline form of Compound-2 with peaks at 1722, 1587, 1578, 1566, 15.37, 1501, 1475, 1456, 1428, 1390, 1359, 1348, 1329, 1319, 1295, 1288, 1276, 1255, 1221, 1199, 1187, 1178, 1165, 1157, 1114, 1058, 1048, 1036,1022, 873, 840, 828, 815, 796, 781, 773, 765, 758, 752, 709, 700, 670, 661,648, 632cm-1.

In another embodiment the present invention provides DSC thermogram of crystalline form of Formula-3 with endothermic peak at 160.18° C.

In another embodiment, the present invention provides a crystalline form of crude Formula- 4 having the X-ray diffraction pattern with peaks at 6.580, 7.586, 9.299, 12.006, 13.172, 13.688, 14.746, 15.197, 17.862, 18.671, 19.054, 19.873, 20.182, 20.877, 22.252, 22.894, 23.201, 23.910, 24.175, 24.822, 25.032, 25.921, 26.530, 26.797, 27.394, 28.242, 29.244, 29.718, 30.710, 31.207, 32.135, 33.368, 34.921, 35.522, 36.170, 36.754, 38.697, 40.513, 43.189, 44.331, 44.907, 45.414, 46.457, 49.512 ±0.2 degrees two theta values.

In another embodiment the present invention provides FTIR spectra of crystalline form of Compound-2 with peaks at 2967, 1850, 1593, 1584, 1546, 1514, 1500, 1485, 1460, 1425, 1392, 1359, 1342, 1303, 1281, 1276, 1253, 1227, 1220 1205 1190,1168, 1133, 1112, 1094, 1084, 1060, 1010, 1004, 949, 901, 882, 868, 849, 815, 782, 771, 724, 699, 678, 630, 605, 587, 576, 566, 552, 545, 533, 517 cm-1.

In another embodiment the present invention provides DSC thermogram of crystalline form of crude Formula- 4 with endothermic peak at 231.17° C.

In another embodiment, the present invention provides a crystalline form of purified Formula-5 having the X-ray diffraction pattern with peaks at 3.357, 6.726, 8.330, 8.735, 9.219, 10.313, 13.560, 15.248, 15.504, 15.990, 16.434, 16.769, 17.550, 17.788, 18.337, 18.647, 19.407, 20.231, 20.716, 21.373, 21.746, 22.577, 22.969, 23.771, 24.253, 24.561, 25.120, 25.731, 26.249, 26.574, 27.138, 27.891, 28.933, 31.748, 32.366, 33.626, 36.043, 37.369, 37.756 ±0.2 degrees two theta values.

In another embodiment the present invention provides FTIR spectra of crystalline form of purified Formula-5 with peaks at 2960, 2152, 1714, 1665, 1620, 1606, 1578, 1520, 1501, 1477, 1405, 1370, 1342, 1299, 1256, 1187, 1172, 1154, 1142, 912.78, 827, 802, 751, 740, 716, 706, 659, 625, 608, 570 cm-1.

In another embodiment the present invention provides DSC thermogram of crystalline form of purified Formula-6 with endothermic peak at 150.97° C.

In another embodiment, the present invention provides a crystalline form of purified Formula-6 having the X-ray diffraction pattern with peaks at 3.357, 6.726, 8.330, 8.735, 9.219, 10.313, 13.560, 15.248, 15.504, 15.990, 16.434, 16.769, 17.550, 17.788, 18.337, 18.647, 19.407, 20.231, 20.716, 21.373, 21.746, 22.577, 22.969, 23.771, 24.253, 24.561, 25.120, 25.731, 26.249, 26.574, 27.138, 27.891, 28.933, 31.748, 32.366, 33.626, 36.043, 37.369, 37.756 ±0.2 degrees two theta values.

In another embodiment the present invention provides FTIR spectra of crystalline form of purified Formula-6 with peaks at 2960, 2152, 1714, 1665, 1620, 1606, 1578, 1520, 1501, 1477, 1405, 1370, 1342, 1299, 1256, 1187, 1172, 1154, 1142, 912.78, 827, 802, 751, 740, 716, 706, 659, 625, 608, 570 cm-1.

In another embodiment the present invention provides DSC thermogram of crystalline form of purified Formula-6 with endothermic peak at 150.97° C.

The present invention relates to the discovery of stable acid addition salts of Bosentan. These acid addition salts are useful for the purification of Bosentan base. The Bosentan acid addition salts of the invention are made from fairly mild acids having a pKa of more than 3. The “pKa” refers to the pKa of the starting acid; hence as used herein reference to the pKa even in the context of the addition salt is referring to the pKa of the starting acid. Suitable acids include, for example, tartaric acid and citric acid.

The Bosentan acid addition salts of the invention are isolatable in a solid state, which can be advantageous. The “solid state” obtained is crystalline. Generally the acid addition Bosentan salts of the invention can be obtained in a stable solid state form making them useful for purification, bulk storage.

The Bosentan acid addition salts of the present invention are typically monovalent salts, i.e., having an acid: base ratio of about 1:1. Analytical methods, such as titration or ionic chromatography, may show a ratio of acid: base of 0.8:1 to 1:1.2 in the isolated solid form of the salt as a result of, e.g., traces of unbound acid and/or base and inherent variance associated with the analytical method. Such variation in the acid: base ratio is encompassed by an acid: base ratio of “about 1:1.”

Exemplary Bosentan acid addition salts according to the present invention include Bosentan citrate and Bosentan tartarate. In a preferred embodiment, the Bosentan acid addition salts of the present invention are Bosentan citrate and Bosentan tartarate. Each of these salts is isolatable in a crystalline solid state with a molar ratio of Bosentan to acid moieties of about 1:1.

The Bosentan acid addition salts of the present invention can be made by combining Bosentan base and an acid having a pKa higher than 3 in an organic solvent, preferably a polar organic solvent, to form a solution, and then precipitating a Bosentan acid addition salt from said solution. Optionally the precipitated Bosentan acid addition salt can be isolated.

A molar equivalent or a slight excess of the starting acid with reference to the Bosentan base is typically used in order to form a Bosentan acid addition salt having an acid: base ratio of about 1:1.

The Bosentan base used in forming the Bosentan acid addition salt (i.e., the starting Bosentan base) is amorphous, in any degree of purity. The starting Bosentan base can also be crude Bosentan that is present in the reaction mixtures obtained after the chemical synthesis of Bosentan.

The organic solvent used is typically a polar organic solvent, which includes both protic and aprotic solvents. Generally, the dielectric constant of a solvent provides a rough measure of a solvent's polarity; solvents with a dielectric constant of less than 15 are typically considered nonpolar. Examples of suitable polar solvents include C3-C10 aliphatic ketones (e.g., acetone, methyl ter.butyl ketone, etc.), C1-C6 chlorinated hydrocarbons (e.g., dichloromethane), C1-C6 aliphatic alcohols (e.g., methanol, ethanol, isopropanol), C3-C10 aliphatic esters (e.g., ethyl acetate), C2-C5 aliphatic nitriles (e.g., acetonitrile), and ethers, including cyclic ethers (e.g., di-isopropyl ether, tetrahydrofuran), as well as mixtures thereof.

There is no specific order in which the Bosentan base and the acid must be combined in the solvent to form the solution. Generally the conditions are such that all of the Bosentan (and all of the acid) is dissolved in the solvent, though strictly speaking such is not required; i.e., some amount of solid or immiscible Bosentan may be present in the solution. The dissolution of Bosentan base in the solvent is advantageously performed at an enhanced temperature, which includes the reflux temperature of the solvent. The contacting or combining of the Bosentan-containing solvent with the acid is advantageously performed at an ambient or higher than ambient temperature, including the reflux temperature of the solvent. In other embodiments, the acid can be added, e.g., substantially at the same time as the base, before the base, etc.

The precipitation of the Bosentan acid addition salt can be carried out in various ways. For example, the precipitation can occur spontaneously upon the contacting of the Bosentan with the acid in the organic solvent. Precipitating of the Bosentan acid addition salt can also be induced by seeding the solution, cooling the solution, evaporating at least part of the solvent, adding an antisolvent, and by combining one or more of these techniques.

The precipitated Bosentan acid addition salt can be isolated from the solution by conventional techniques, e.g. filtering or centrifugation, and can be washed and dried.

The isolated Bosentan acid addition salt can, however, be purified if desired. For example, the isolated salt is recrystallized or reprecipitated by dissolving (at least partially, e.g., suspending) the isolated salt in a solvent, such as any of the above defined polar organic solvents, at an enhanced temperature (which includes a reflux temperature of the solvent), and then crystallizing or precipitating the salt from the solvent. The recrystallization (reprecipitation) process may be repeated until a desired purity of the isolated Bosentan acid addition salt is obtained. For clarity, the terms “purify,” “purification,” “purified,” and variations thereof are used herein to indicate an improvement in the quality or purity of the substance and are not meant in the narrow sense of obtaining near absolute purity. Hence reducing the impurities from 2.0% to 1.5% represents a “purification” of the substance.

The solid state Bosentan acid addition salts of the present invention can be advantageously used to obtain purified Bosentan. In general, crude Bosentan can be purified by converting it to a Bosentan acid addition salt as described above and then converting the Bosentan salt back into Bosentan base. For example, a purification process can comprise (i) combining crude Bosentan and an acid having a pKa more than 3 in a first solvent or solvent mixture, preferably from polar organic solvents, to obtain an acid addition salt of Bosentan; (ii) isolating the acid addition salt of Bosentan in solid state from the first solvent or solvent mixture; (iii)preferably purifying the acid addition salt of Bosentan using second solvent or solvent mixture till the required purity is attained(iv) converting the Bosentan acid addition salt into Bosentan base in a third solvent, preferably an polar organic solvent in presence of a base such as alkali and alkali metal hydroxides alkali metal carbonate; and (iv) isolating the Bosentan base from said third solvent by evaporation to get amorphous Bosentan. Because structurally related impurities present in the crude Bosentan are generally soluble in the organic solvents used to form the salt, these impurities generally remain in the first solution during the isolation of the solid Bosentan acid addition salt; thereby separating these impurities from the Bosentan moiety. The conversion to Bosentan base, especially in an polar organic solvent and water in presence of a base, can likewise provide a further purification effect with respect to water-soluble impurities. “Crude Bosentan” means a Bosentan base or salt having insufficient purity and includes reaction mixtures obtained after the chemical synthesis of Bosentan as well as Bosentan having near pharmaceutical grade purity. From a practical standpoint, the crude Bosentan is typically a Bosentan base in amorphous form. Likewise, the produced “Bosentan base,” which has an enhanced purity or quality relative to the crude Bosentan, is also amorphous. The above-recited process steps are not exhaustive; additional steps can also be included. For example, the acid addition salt of Bosentan can itself be purified, such as by (re)crystallization as described above, before being converted to Bosentan base.

The first solvent is generally a polar organic solvent as described above in the context of making the Bosentan acid addition salts. Thus, examples of suitable first solvents include C3-C10 aliphatic ketones (e.g., acetone, methyl ter.butyl ketone, etc.), C1-C6 chlorinated hydrocarbons (e.g., dichloromethane), C1-C6 aliphatic alcohols (e.g., methanol, ethanol, isopropanol), C3-C10 aliphatic esters (e.g., ethyl acetate), C2-C5 aliphatic nitrites (e.g., acetonitrile), and ethers including cyclic ethers (e.g., di-isopropyl ether, tetrahydrofuran), as well as mixtures thereof.

The Bosentan acid addition salt, which can be formed before or during precipitation thereof, is conveniently isolated as a solid from the first solvent by known techniques such as filtration, etc. The precipitation of the solid state acid addition salt of Bosentan can be carried out by the techniques as described above.

The isolated solid Bosentan acid addition salt can be converted into Bosentan base by any suitable or convenient technique. Generally, the solid salt is dissolved in the second solvent and converted to base, preferably via the use of a base. Advantageously the second solvent is an polar solvent in which Bosentan base is soluble. Such solvents include water-immiscible solvents and combinations thereof. The base used to convert the salt of Bosentan to Bosentan base may be an organic or inorganic base and is preferably a base that binds the acid present in the second solvent to form a salt that is soluble in the second solvent. Suitable bases include sodium and potassium hydroxide, TEA. Upon addition of the base to the salt-containing second solvent, Bosentan generally gets extracted in the organic solvent and directly converted to amorphous polymorph upon concentration and drying.

In a preferred embodiment, the above purification process results in Bosentan base having less than 0.5% impurities.

The present invention provides a method for the preparation of compound of Formula-1 according to the known processes in the prior art.

The invention is further illustrated by the following examples, which should not be construed to limit the scope of the invention in anyway.

Characterization

Bosentan intermediate of the present invention (Formula -2), (Formula-3), (Formula-4), (Formula -5), (Formula -6) is characterized by X-Ray powder Diffraction (XRD), DSC analysis, and FTIR spectroscopy.

XRD: XRD Diffractograms were collected on Bruker AXS D-8 advance X-Ray powder diffract meter, Scintillation detector. Scanning Parameters: ScanType—Locked Coupled, Scan Mode—Continuous, Range (2 theta)—3.0°-60.0°, Rate—3.6°/min

FTIR Spectroscopy

FTIR Spectrum was recorded on Perkin-Elmer spectrum-1 spectrometer, Diffuse Reflectance Technique. The sample was finely ground with Potassium Bromide, and the spectrum was recorded using Potassium Bromide background in a Diffused reflectance accessory.

Thermal Analysis

Differential Scanning calorimetry was performed on Perkin Elmer Diamond. The Crucible was Crimped and punched prior to analysis. Experimental conditions: sample Weight: 2.0-3.0 mg, Heating Rate: 10° C./min.

The present disclosure is further elaborated with the help of following examples and associated figures. However, these examples should not be construed to limit the scope of the present disclosure.

### EXAMPLES

### Example 1

### Preparation of 5-(2-methoxyphenoxy) [2,2]-bipyrimidinyl diol (Formula-2)

To a mixture of methanol (60 mL) and sodium methoxide (3.8 g, 0.07 mol) under inert atmosphere pyrimidine -2-carboxyamidine hydrochloride (3.3 g, 0.02 mol) was added followed by 2-(2-methoxy phenoxy) malonic acid diethyl ester (5 g, 0.02 mol) at the ambient temperature and stirred for 3 h. Water was added to the concentrated reaction mass and the pH was adjusted to 2 using 1N hydrochloric acid and stirred for 1 h at the ambient temperature and stirred for 2h at 5-10° C. The obtained solid was filtered and washed with water and dried under vacuum for 10 hours yielding compound-2 (4.0 g) as a pale yellow crystalline solid (99%) having the X-ray diffraction pattern with peaks at 11.062, 11.516, 12.523, 14.886, 16.081, 16.368, 17.12, 18.04, 18.286, 18.994, 19.697, 20.74, 21.359, 22.592, 23.232, 24.075, 25.099, 25.767, 26.054, 26.645, 27.343, 28.063, 29.074, 30.323, 31.449, 33.016, 34.653, 36.016, 36.708, 37.136, 38.393, 41.271, 42.419, 44.641, 46.637, 49.058, 49.468, 50.041, ±0.2 degrees two theta values.

### Example 2

### Preparation of 4,6- dichloro 5-(2-methoxyphenoxy) [2,2]-bipyrimidinyl compound (formula-3)

5-(2-methoxyphenoxy) [2,2]-bipyrimidinyl diol (Formula-2) 4 g (0.0128 mol) was taken in acetonitrile (20 mL) and Collidine (3.88 g, 0.032 mol). Phosphorous oxy chloride was added (19.64 g, 0.128 mol) slowly and refluxed for 5 to six hours, the reaction mass were quenched in water and the product was extracted in ethyl acetate. Organic layer was washed with water, sodium bicarbonate and saturated brine and dried over anhydrous sodium sulphate and concentrated. The concentrated reaction mass was isolated using n-heptane and the obtained solid was dried under vacuum yielding 3.4 g of slightly brown solid 4,6- dichloro 5-(2- methoxyphenoxy) [2,2]-bipyrimidinyl compound (formula-3) (99%) having the X-ray diffraction pattern with peaks at 8.547, 9.867, 11.068, 11.97, 13.851, 14.726, 15.539, 17.213, 18.428, 20.463, 22.232, 22.704, 23.536, 23.937, 24.827, 26.291, 26.545, 27.294, 27.815, 28.223, 29.278, 30.022, 30.5, 31.447, 31.996, 32.454, 33.43, 33.644, 34.251, 34.89, 35.662, 36.393, 37.402, 38.523, 39.022, 40.238, 40.724, 41.35, 41.93, 43.732, 44.289, 45.24, 47.267, 47.978, 49.123, 49.438, 50.22, ±0.2 degrees two theta values.

### Example 3

### Preparation of 4-tert-Butyl-N-[6-chloro5-(2-methoxyphenoxy) [2,2]- bipyrimidinyl-4-yl]benzene sulfonamide compound of formula-4.

p-t-butyl benzene sulphonamide (2.2 g, 0.0104 mol) was taken in Dimethyl Sulphoxide and potassium carbonate was added (2.75 g, 0.0198 mol) under inter atmosphere and stirred for 0.5 h. 3.4 g of 4,6- dichloro 5-(2-methoxyphenoxy) [2,2]-bipyrimidinyl compound (formula-3) 3.44 g, 0.009 mol was added and heated to 120° C. and maintained at the same temperature for 2 h. The reaction mass was quenched in 1N hydrochloric acid and stirred for 2 h. Product precipitates out and was filtered and washed with water and dried under vacuum yielding 4.3 g (96%) of pale yellow crystalline solid 4,6- dichloro 5-(2-methoxyphenoxy) [2,2]- bipyrimidinyl compound of formula-3 having the X-ray diffraction pattern with peaks at 8.547, 9.867, 11.068, 11.97, 13.851, 14.726, 15.539, 17.213, 18.428, 20.463, 22.232, 22.704, 23.536, 23.937, 24.827, 26.291, 26.545, 27.294, 27.815, 28.223, 29.278, 30.022, 30.5, 31.447, 31.996, 32.454, 33.43, 33.644, 34.251, 34.89, 35.662, 36.393, 37.402, 38.523, 39.022, 40.238, 40.724, 41.35, 41.93, 43.732, 44.289, 45.24, 47.267, 47.978, 49.123, 49.438, 50.22, ±0.2 degrees two theta values.

This was optionally purified using acetonitrile under the reflux condition to give pure pale yellow to off white crystalline product for formula-4 having the XRD.

### Example 4

### Preparation of Bosentan

Mono sodium ethylene glycolate was dissolved in ethylene glycol 26.72 g, 0.430 mol and 4- tert-Butyl-N-[6-chloro5-(2-methoxyphenoxy) [2,2]-bipyrimidinyl-4-yl]-benzene sulfonamide 3.7 g (0.007 mol) was added and heated to 110 to 115° C. for 5 h. The reaction mass was quenched with 141 ml water and then acidified to pH 2 using 1N hydrochloric acid and the product was extracted in Dichloro methane. Organic layer was dried over anhydrous sodium sulphate and concentrated to get 3.5 g of crude amorphous Bosentan.

### Example 5

### Preparation of Bosentan Citrate

Bosentan 1.0 g, 0.0018 mol and citric acid 0.35 g, 0.0018 mol were taken in mixture of acetonitrile 4 mL and stirred at an ambient temperature for 8 h and 8 ml of diisopropyl ethyer was added. The reaction mixture was stirred for 30 min and the solid was filtered and washed with di isopropyl ether 2.0 mL to get 99.0% pure material and was further purified using ethylacetate acetonitrile and Diisopropyl ether mixture. Bosentan citrate having the X-ray diffraction pattern with peaks at 3.357, 6.726, 8.330, 8.735, 9.219, 10.313, 13.560, 15.248, 15.504, 15.990, 16.434, 16.769, 17.550, 17.788, 18.337, 18.647, 19.407, 20.231, 20.716, 21.373, 21.746, 22.577, 22.969, 23.771, 24.253, 24.561, 25.120, 25.731, 26.249, 26.574, 27.138, 27.891, 28.933, 31.748, 32.366, 33.626, 36.043, 37.369, 37.756 ±0.2 degrees two theta values.

### Example 6

### Preparation of Bosentan Tartarate

Bosentan 1.0 g, 0.0018 mol and tartaric acid 0.27 g, 0.0018 mol were taken in mixture of acetonitrile 4 mL and stirred at ambient temperatures for 8 h and 8 ml of diisopropyl ethyer was added. The reaction mixture was stirred for 30 min and the solid was filtered and washed with di isopropyl ether 2.0 mL to get 99.0% pure material and further purified using ethylacetate acetonitrile and Diisopropyl ether mixture. Bosentan citrate having the X-ray diffraction pattern with peaks at 3.416, 6.799, 8.933, 9.260, 9.845, 10.639, 11.564, 14.224, 15.461, 16.194, 16.882, 17.618, 17.843, 18.266, 18.456, 19.421, 20.140, 20.594, 21.420, 22.463, 23.338, 23.830, 24.567, 25.063, 26.500, 27.802, 29.161, 29.677, 31.528, 31.998, 33.435, 36.137, 36.644, 37.486, 39.437, 42.596, 45.021, 46.681, 47.031, 47.730, 49.188, 51.536, 57.573, 59.858 ±0.2 degrees two theta values.

### Example 7

### Preparation of Bosentan Base

Bosentan citrate 1.0 g, 0.00134 mol and methylene di chloride 8 ml were taken in water 8 ml and the pH was adjusted to 8 using 1% sodium bicarbonate solution. The mass was stirred at an ambient temperature for 1 h and separated.

We claim:

1. Acid addition salts of Bosentan.

2. The Bosentan acid addition salt according to claim 1, wherein said acid has a pKa higher than 3.

3. The Bosentan acid addition salt according to claim 1, wherein said salts are in solid form.

4. The Bosentan acid addition salt according to claim 3, wherein said salts are in crystalline form.

5. The Bosentan acid addition salt according to claim 1, wherein said acid addition salt is selected from Bosentan citrate and Bosentan tartarate.

6. The Bosentan acid addition salt according to claim 5, wherein said salt is selected from crystalline Bosentan citrate and crystalline Bosentan tartarate.

7. A method of making an acid addition salt of Bosentan, which comprises: a) combining Bosentan base and an acid having a pKa higher than 3 in an organic solvent to form a solution; b) precipitating a Bosentan acid addition salt from said solution and c) isolating the precipitated Bosentan acid addition salt.

8. The method according to claim 7, wherein said acid is selected from the group of citric acid and tartaric acid.

9. The method according to claim 7, wherein said organic solvent is selected from the group consisting of ketones, chlorinated hydrocarbons, hydrocarbons, alcohols, esters;
aliphatic nitriles; ethers and mixtures thereof

10. The method according to claim 9, wherein said organic solvent is selected from the group consisting of acetone, methyl tert.butyl ketone, dichloromethane, methanol, ethanol, isopropanol, ethyl acetate, butyl acetate, acetonitrile, di-isopropyl ether, methyl tertiary butyl ether tetrahydrofuran, and mixtures thereof

11. A method of purifying Bosentan, which comprises: a) preparation of an acid addition salt of Bosentan by following any method discussed in earlier claims, b) isolation of said acid addition salt of Bosentan from organic solvent c) optionally re crystallizing by using an organic solvent d) converting Bosentan acid addition salt into Bosentan base and e) isolation of Bosentan base.

12. According to claim 11, wherein Bosentan base is amorphous in nature.

13. The method according to claim 12, wherein said organic solvent is selected from the group consisting of ketones, chlorinated hydrocarbons, hydrocarbons, alcohols, esters, aliphatic nitrites, ethers and mixtures thereof.

14. According to claim 13, wherein said organic solvent is selected from the group consisting of acetone, methyl tert.butyl ketone, dichloromethane, methanol, ethanol, isopropanol, ethyl acetate, butyl acetate, acetonitrile, di-isopropyl ether, methyl tertiary butyl ether tetrahydrofuran, and mixtures thereof.

15. The method according to claim 11, wherein said converting step comprises contacting said Bosentan acid addition salt with an organic or inorganic base in organic solvent.

16. The method according to claim 11, which further comprises recrystallizing said isolated acid addition salt of Bosentan prior to said converting step.

17. A process which comprises dissolving a solid Bosentan acid addition salt according to claim 12 in an organic solvent, and precipitating said salt to obtain a purified solid Bosentan acid addition salt.

18. Bosentan citrate salt.

19. Bosentan tartarate salt.
